(19) United States
(12) Patent Application Publication
(10) Pub. No.: US 2021/0383099 A1
(43) Pub. Date: Dec. 9, 2021

(54) THERMAL SENSING AND IDENTITY AUTHENTICATION SYSTEM AND METHOD

(71) Applicant: Barend Oberholzer, West Hollywood, CA (US)

(72) Inventor: Barend Oberholzer, West Hollywood, CA (US)

(73) Assignee: ROYAL HOLDINGS TECHNOLOGIES CORP, West Hollywood, CA (US)

(21) Appl. No.: 16/893,142

(22) Filed: Jun. 4, 2020

(51) Int. Cl. G06K 9/00; G01J 5/10; G16H 40/67; G16H 50/30; G06Q 50/26; G06K 7/14; G07C 9/22

(52) U.S. Cl. CPC G06K 9/00288; G01J 5/10; G06K 9/00281; G16H 40/67; G01J 2005/0077; G06Q 50/265; G06K 7/1417; G06K 7/1413; G07C 9/22; G16H 50/30

(57) ABSTRACT

A thermal sensing and identity authentication system is provided. The system may be used for screening of persons entering a venue or other location. The system may include a mobile device, a mobile application, a backend platform, an image/facial recognition system, a thermal imaging system, a standalone temperature sensing device, and other elements. The system may scan one or more persons (e.g., a crowd of persons) to identify targets (e.g., persons' foreheads) for measurement, and subsequently take thermal imaging measurements of the targets to determine any persons who may have an elevated body temperature. The system may also enable individuals to take their own body temperature measurements, either using the thermal imaging system and/or the temperature sensing device, offsite so that they may not be required to go through onsite screening. In this case, the system may authenticate the person's identity, take his/her body temperature readings, and communicate this information to the venue. Then, upon arrival the venue, the venue may use this information to grant (or deny) the person access.

### COPYRIGHT STATEMENT

This patent document contains material subject to copyright protection. The copyright owner has no objection to the reproduction of this patent document or any related materials in the files of the United States Patent and Trademark Office, but otherwise reserves all copyrights whatsoever.

### FIELD OF THE INVENTION

This invention relates to a framework, system, and method for thermal sensing and identity authentication, including the authentication of body temperature measurements through the use of image recognition information.

### BACKGROUND

Precautionary screening measures are becoming commonplace to regulate access into venues such as businesses, restaurants, concerts, sporting events, hair salons, etc. Common screening measures include taking a person's body temperature to determine if he/she has a fever, requiring that each person wash his/her hands or use alcohol-based hand rub (ABHR) prior to entering, determining if a person has had contact with anyone with a confirmed infectious disease (e.g., COVID 19) in the last 14 days, etc.

In one example, each person wishing to enter a venue may be required to have his/her body temperature taken onsite immediately prior to being granted access into the venue. This may ensure that no one with a fever (and potentially an infectious disease such as COVID 19) is able to enter. However, for large and crowded venues, this may result in long queues and extended wait times for each and every person to be tested.

Accordingly, there is a need for a thermal sensing and identity authentication system that can scan a crowd of people and identify those with potential fevers. There is also a need for a system that enables people to precheck themselves remotely so that onsite screening may be avoided.

### BRIEF DESCRIPTION OF THE DRAWINGS

Other objects, features, and characteristics of the present invention as well as the methods of operation and functions of the related elements of structure, and the combination of parts and economies of manufacture, will become more apparent upon consideration of the following description and the appended claims with reference to the accompanying drawings, all of which form a part of this specification. None of the drawings are to scale unless specifically stated otherwise.

FIG. 1 shows an overview of a thermal sensing and identity authentication system in accordance with exemplary embodiments hereof;

FIG. 2 shows aspects of a thermal sensing and identity authentication system in accordance with exemplary embodiments hereof;

FIG. 3 shows an example thermal image in accordance with exemplary embodiments hereof; and;

FIGS. 4-9 show aspects of a thermal sensing and identity authentication system in accordance with exemplary embodiments hereof;

FIG. 10 shows aspects of a thermal sensing and identity authentication system computing environment in accordance with exemplary embodiments hereof; and

FIG. 11 depicts aspects of computing and computer devices in accordance with exemplary embodiments hereof.

### DETAILED DESCRIPTION OF EXEMPLARY EMBODIMENTS

As used herein, unless used otherwise, the following terms and abbreviations have the following meanings:

API means application programming interface.

SDK means software development kit.

“Screening” generally refers to any activity that collects any type of information from a particular person or persons for the purpose of identifying whether the person or persons are at risk to themselves and/or others. For example, screening may involve authenticating a person's identity, taking a person's body temperature to determine whether or not the person has a fever (and potentially an infectious disease such as COVID 19), requiring that the person answer one or more questions such as if he/she has been in contact with any at-risk persons in the last 14 days or if he/she is experiencing shortness of breath or a cough, and other types of screening.

“Screening information” generally refers to any type of data, measurements and/or other information gathered during a screening. For example, screening information may include a person's identity, a person's body temperature (to confirm that the person does not have a fever), answers to questions regarding whether or not a person has been in contact with any at-risk persons in the prior 14 days, answers to questions regarding whether or not the person is experiencing shortness of breath, a cough, chills, muscle pain and/or other symptoms that may indicate that the person may be at risk of a viral disease, and other types of information.

In general, the system according to exemplary embodiments hereof, provides a system and method that screens one or more people to detect elevated body temperatures. In some aspects, the system may scan a crowd of people to identify persons within the crowd with potentially elevated temperatures. In other aspects, the system may enable mobile offsite screening of one or more persons so that onsite screening may be avoided.

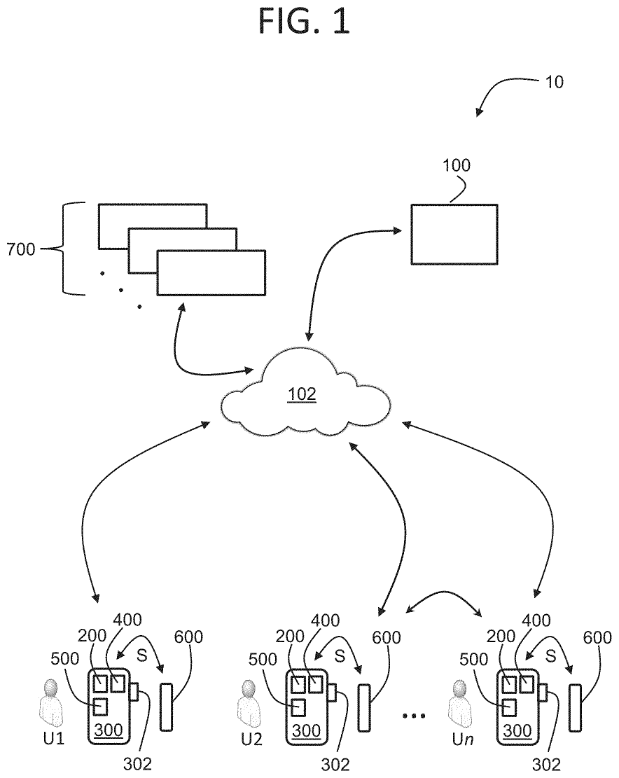

FIG. 1 shows an overview of an exemplary framework for a thermal sensing and identity authentication system 10 (also referred to herein as simply the system 10) according to exemplary embodiments hereof. As shown, the thermal sensing and identity authentication system 10 may include a backend controller 100 that may interface with users U1, U2, . . . Un of the system 10 (individually and/or collectively) via one or more application interfaces 200 (e.g., a mobile application or “app”, a browser, website or Internet interface, or other types of applications) running on one or more computing devices 300 (e.g., smart phones, tablet computers, laptops, desktop computers, mobile media players, etc.). The system 10 also may include an image recognition system 400, a thermal imaging system 500 and a temperature sensing device 600. The system 10 also may include other systems, elements and components as required by the system 10 to fulfill its functionalities. In addition, the system 10 may interface with various external systems 700 (e.g., businesses, venues, events, etc.).

The computing devices 300 and the backend controller 100 may preferably be connected to one or more networks 102 (e.g., the Internet, LAN, WAN, wireless communication systems, cellular communication systems, telephony or other types of communication systems or protocols) and may communicate thereby. In some embodiments, the backend controller 100 may include a cloud platform (e.g., one or more backend servers), one or more local controllers, or any combination thereof. In some embodiments, the backend controller 100 includes a cloud platform that interfaces with one or more local controllers. For example, administrators An of the system 10 may interface with the system 10 via a local controller in communication to a cloud platform.

In some embodiments, the application 200 includes a mobile application (“app”) running on a mobile device 300 (e.g., a mobile device 300 integrated with other elements of the system, a user's mobile device 300, and/or other mobile devices). The application 200 may provide a graphical user interface (GUI) that enables the user Un to interface with the application 200, the backend 100 and the overall system 10. The application 300 may generally provide an interface with which the user Un may enter information for the system 10 to utilize (e.g., upload to the backend 100), and interface controls (e.g., touchscreen buttons, etc.) for the user Un to activate while interacting with the system 10. The application 300 also may display data and other types of information that the user Un may read or otherwise consume (e.g., body temperature readings of a target and/or of the user Un himself/herself). In general, and in some embodiments, the application 200 may provide a primary interface with which the user Un may interact with the system 10.

In some embodiments, the image recognition system 400 operates as a general image recognition system as well as a facial image recognition system. Accordingly, the system 400 may include an image recognition application 402 and/or a facial recognition application 404 running on the user's device 300 and/or the backend 100. In some embodiments, the image recognition application 402 and the facial recognition application 404 may be combined.

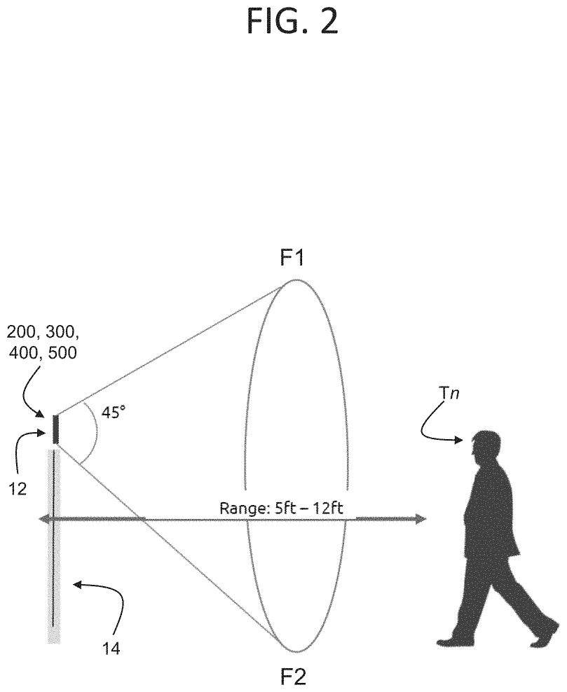

The image recognition system 400 interfaces with the device's camera 302 to receive images to be processed (e.g., recognized). For example, as shown in FIGS. 2-4, the image recognition system 400 may be used to view a user's face and/or a temperature sensing device 600 in order to recognize one and/or the other. It may be preferable that the user's face and/or the temperature sensing device 600 be within the camera's field of view (shown as field of view lines F1 and F2) so that the camera 302 may provide images of the user's face and/or the device 600 of sufficient clarity to the system 400.

In some embodiments, the image recognition application 402 and/or the facial recognition application 404 includes a native application running on the user's device 300 (e.g., provided with the device 300). In other embodiments, the image recognition application 402 and/or the facial recognition application 404 is included as part of the device's operating system (OS) or firmware. In other embodiments, the image recognition application 402 and/or the facial recognition application 404 is at least partially integrated into the application 200. In other embodiments, the image recognition application 402 and/or the facial recognition application 404 includes a standalone application, or any combination of the above. For the purposes of this specification, the image recognition application 402 and/or the facial recognition application 404, unless otherwise stated, will be considered to be integrated and/or controlled by the application 200 such that reference to the application 200 also implies reference to the image recognition application 402 and/or the facial recognition application 404 where applicable.

In some embodiments, the facial recognition application 404 uses biometrics to map facial features from a captured facial image (e.g., from the camera 302) and then compares the information with a database of known faces to find a match and to thereby authenticate the user's identity.

In one example, the facial recognition application 404 may perform one or more acts, including without limitation:
1. Detect, track and score facial images from live video or images (e.g., from the camera 302);
2. Create biometric templates of the best images of faces for comparison to known faces;
3. Compare the biometric template(s) to known faces within one or more databases (in the cloud or otherwise); and
4. Find a positive match and assist in correlating the determined identity with other information (e.g., body temperature information).

In one exemplary embodiment hereof, the image recognition system 400 may implement machine learning (e.g., a machine learning kit library) to detect a face and produce face coordinates for cropping. In this way, the system 10 may create a facial image. The system 10 may then score the detected faces and select the facial images that may include the best resolution. These images may then be sent to the cloud platform 100 for face recognition. The cropped face (preferably about ˜100 klb in file size) may be sent to the cloud platform for conversion to a biometric template and the image may be matched (e.g., the identity of the facial image may be identified). The identified face information may then be sent back to system 10 and correlated with other information (e.g., body temperature information). It may be preferable that this entire process take about <1 second.

In some implementations, the facial recognition system 400 may identify the identity of the subject, the gender of the subject, the age of the subject, the ethnicity of the subject, facial characteristics of the subject (e.g., glasses, beard, eye/hair color, eyes open/closed, etc.), and the sentiment and emotional state of the subject (e.g., happy/smiling, sad, angry, nervous, etc.). However, it may not be necessary for the facial recognition system 404 to identify all of the above attributes.

In some embodiments, the system 10 includes a thermal imaging system 500 as described in U.S. patent application Ser. No. 16/436,752, filed Jun. 10, 2019, the entire contents of which are hereby fully incorporated herein by reference for all purposes.

In one exemplary embodiment hereof, the thermal imaging system 500 may include an infrared camera 502 that may detect energy (e.g., heat), convert the energy to an electronic signal, and then send the electronic data to the application 200, mobile device 300, backend 100 and/or other systems for processing. The result may include a calculated temperature of the target and/or a thermal image of the target representing the calculated temperature.

In some embodiments, the system 10 includes a thermal imaging system 500 as described in U.S. patent application Ser. No. 16/436,752, filed Jun. 10, 2019, the entire contents of which are hereby fully incorporated herein by reference for all purposes.

In one exemplary embodiment hereof, the temperature sensing device 600 includes a standalone thermometer and/or other type of temperature sensing device. In some embodiments, the device 600 is a digital device capable of interfacing (pairing) with the mobile device 200 via Bluetooth®, Wi-Fi, cellular communications, telephony, infrared (IR), other types of communication protocols and/or any combination thereof. The device 600 may typically include a housing, a thermistor, a microcontroller, pairing electronics, and optionally, an electronic display. During use, the temperature sensing device 600 is placed under the tongue of the user Un (or optionally, under the user's armpit). A temperature reading is taken and processed to reflect the user's body temperature. It is understood however that other types of temperature sensing devices 600 with other components also may be used and that the scope of the system 10 is not limited in any way by the type of temperature sensing device 600 employed.

### Broad Thermal Surveillance

In some exemplary embodiments as shown in FIG. 2, the computing device 300 (e.g., a smartphone running the application 200), the image recognition system 400 and the thermal imaging system 500 are integrated as a combined single unit 12 that may interface with the backend 100 as well as other systems. The unit 12 may be mounted on a tripod 14 (or similar) and/or may be handheld. Note that some aspects of the elements 400, 500 may reside on the computing device 300, the backend 100 or any combination thereof.

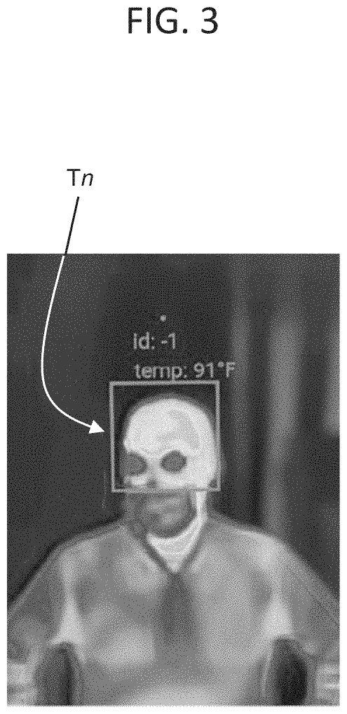

In some embodiments, the unit 12 is adapted to perform at least some of the following functionalities:
1. Using the image recognition system 400 and/or the thermal imaging system 500, scan one or more persons (e.g., a crowd of persons) within a scanning area. In one example, this may include scanning a crowd of people entering a venue.
2. Using the image recognition system 400 (the facial recognition application 402), recognize applicable targets Tn within the crowd. The system 402 may first capture and process an image of a person's face and then identify the forehead region of the face as the thermal reading target Tn.
3. Using the thermal imaging system 500, take thermal readings of the targets Tn (e.g., of each person's forehead as he/she passes through the scanning area). An example of this is shown in FIG. 3.
4. Using the computing device 200, process the thermal readings to determine if one or more of the persons has an elevated body temperature.
5. Using the image recognition system 400, identify the identity of the one or more persons with elevated body temperatures; and
6. Using the computing device 200 and/or the backend 100, alert the appropriate personnel to provide further assistance (e.g., intercept the identified persons and implement secondary screenings).

In some embodiments, as shown in FIG. 2, the scanning range between the unit 12 and the target Tn may be about 5 feet to 12 feet. In other embodiments, the scanning distance may be about 5 feet to 24 feet. In some embodiments, the scanning range resembles a deep conical shape as shown. In this way, the system 10 may scan persons of interest for elevated temperatures without making physical contact with the persons and/or without having to overly slow the general flow the persons' foot traffic speed.

In some embodiments, the unit 12 and the system 10 (e.g., the thermal imaging system 500) measures the skin temperature of the forehead region of a target person. As is known in the art, the skin temperature of a person's forehead has a direct correlation with the core temperature of the individual. This relationship is based on the temporal artery and its location in close proximity in two areas of the face, the inner eyelid, and the forehead. For example, a forehead temperature reading of about 92° F. (33.3° C.) implies a core body temperature of the individual of about 98.1° F. (36.7° C.), which is a non-fever status. In another example, a skin temperature of about 98° F. (36.7° C.) implies an elevated core body temperature of about 100.5° F. (38.1° C.) indicating a potential fever.

In another embodiment, the system 10 may be used by a particular user Un to identify his/her face and forehead, and to take an associated thermal imaging reading to determine his/her body temperature. This is shown in FIG. 3. As shown, the person may hold his/her mobile device 300, equipped with the app 200, the facial recognition system 400 and the thermal imaging system 500, such that the cameras 302, 402 may view his/her face (shown as field of view lines F1 and F2). The system 10 may identify the user's forehead FH and take an associated thermal imaging reading T_{F}. The application 200 and/or the backend 100 may then process the information and display the resulting body temperature to the user Un.

### Temperature Measurement with Identity Authentication

In another embodiment hereof, the system 10 provides a system and method of determining the body temperature of a user Un while simultaneously authenticating the same user's identity. In this way, the system 10 may correlate the temperature reading and the identity of the user Un, and this information may be utilized as screening information to grant the user Un access to a particular venue and/or for other purposes. In some embodiments, the system 10 is mobile such that the screening may take place offsite thereby enabling the user Un to bypass the onsite temperature-taking queues and be granted access to enter the venue directly.

For example, in some embodiments, the user Un may utilize his/her own personal mobile device 300 (e.g., smartphone) equipped with the necessary elements of the system 10 (e.g., the applications 200 and/or 400, the temperature sensing device 600, etc.) to take his/her own body temperature reading and to authenticate his/her identity prior to arrival at the venue. The system 10 may communicate the temperature readings along with the authenticated identity of the user Un to the venue, and the user Un may be granted access upon arrival without having to be screened onsite. This saves the user time (no waiting in line at the venue) and the venue time and money (less personnel having to prescreen attendees).

In some embodiments, the system 10 is adapted to interface with (and/or be integrated with) the screening and access system(s) 700 of one or more venues so that the overall process is seamless. For example, the system 10 may be adapted to interface with an employee badge swiping system 700 such that when an authenticated user Un swipes his/her badge for admission, the badge system 700 acknowledges his/her identity and body temperature reading and allows (or denies) the user Un access. The system 10 also may be adapted to interface with various systems 700 in other ways (e.g., may require at least some manual involvement and/or intervention).

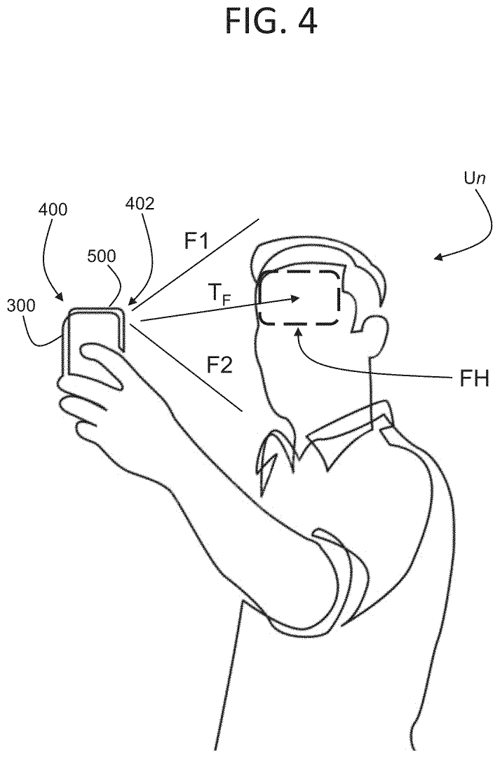

In some exemplary embodiments hereof as shown in FIG. 1 and FIG. 4, the mobile device 300 (via application 200) interfaces with the temperature sensing device 600. This is denoted by the arrow S. In some embodiments, each temperature sensing device 600-1, 600-2, 600-3, . . . 600-n includes a unique identifier (e.g., a serial number, an IP address, etc.) that the mobile device 300 (e.g., the application 200) identifies during the communication. In this way, the application 200 and/or 400 may identify and authenticate the sensing device 600 during its use.

In some embodiments, the mobile device 300 (e.g., the application 200) includes drivers and/or other types of software that it may use to generally control the device 600. For example, the mobile device 300 may trigger the device 600 to take temperature readings, may request and receive the measurement data from the device 600, may reset the device 600, may calibrate the device 600, and may generally control the device 600 in any way as necessary during its use. At the same time (preferably simultaneously or just prior), the facial recognition system 404 may authenticate the user's identity. The system 10 may then correlate the user's identity with the temperature sensing device's unique identifier and the measured temperature readings. In this way, the temperature readings are authenticated as coming from the particular sensing device 600 and for the particular user Un.

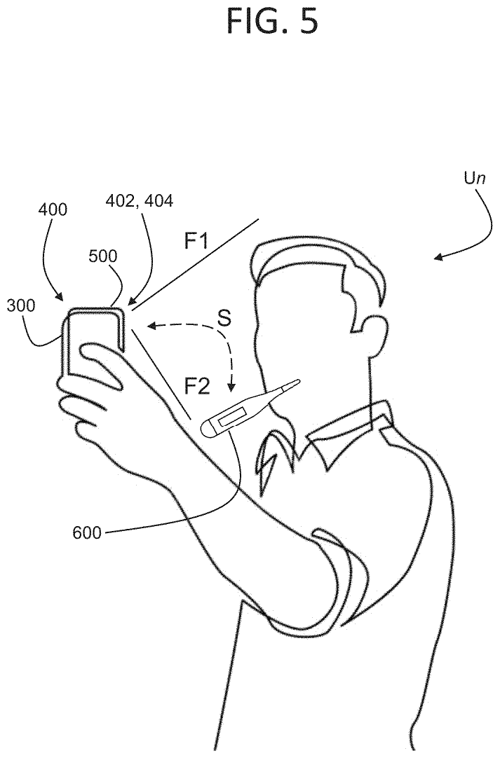

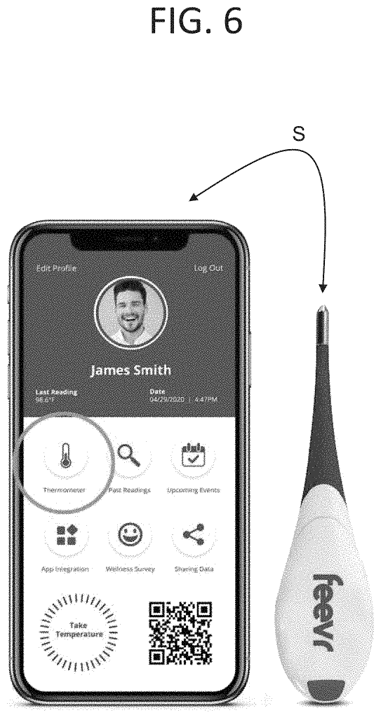

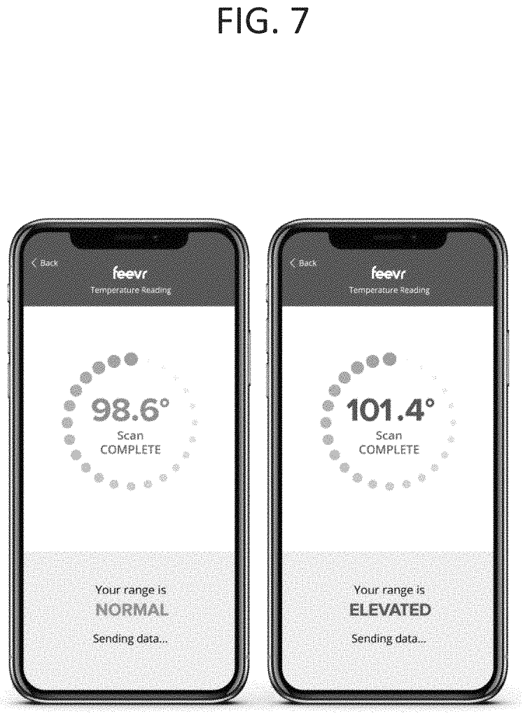

To provide a general understanding of this procedure, a summary of steps is described below.
1. First, the user Un launches the application 200 on the mobile device 300 and pairs the mobile device 300 with the temperature sensing device 600. This is shown in FIG. 6.
2. The mobile application 200 communicates with the sensing device 600 and acquires its identifier (e.g., serial number);
3. Next, the user Un places the temperature sensing device 600 into his/her mouth (or under his/her armpit). See FIG. 5.
4. The user Un then holds the mobile device 300 with its camera 302 pointed towards his/her face (see FIG. 2). See FIG. 5. In some embodiments, the user Un may be required to maintain this position for up to 15 seconds while this entire process runs.
5. Next, the user Un instructs the application 200 to take a temperature reading (note that this may be triggered automatically by the application 200 once it confirms that it can sufficiently view the user's face);
6. Next, the facial recognition system 400 views the user's face and identifies the user's identity;
7. Upon identifying the user Un, the application 200 triggers the temperature sensing device 600 to take one or more temperature readings;
8. Upon taking the temperature readings, the device 600 communicates the readings to the application 200 and the application 200 determines whether or not the user Un has an elevated body temperature (e.g., a fever).
9. The system 10 then provides this information to the user Un (e.g., on the GUI of the application 200 as shown in FIG. 7) and/or relays the screening information to any applicable entities for decision making purposes (e.g., whether or not to grant the user Un access to a particular venue). Note that in this case, the screening information may include the authenticated user's identity, the user's body temperature and the date and time of the temperature reading.

One issue that may exist includes a first user U1 with a first temperature sensing device 600-1 in his/her mouth, and a second user U2 with a second sensing device 600-2 in his/her mouth. If the system 10 is paired with the second sensing device 600-2 but visually authenticates the first user U1, the system 10 may inadvertently trigger and take temperature readings of the second user U2 using the second sensing device 600-2 and attribute the readings to the first user U1. This may purposely or inadvertently circumvent the accuracy of the system 10.

To solve this issue, in one exemplary embodiment hereof, the image recognition system 400 also may identify other objects, and in particular, may visually identify and authenticate a particular temperature sensing device 600 that a user Un is using to take his/her temperature. This may ensure that the temperature sensing device 600 in communication with the system 10 (the app 200) is the same sensing device 600 in the user's mouth (or armpit). This may occur at the same moment that the facial recognition system 404 identifies (i.e., authenticates) the user's identity. In this way, the system 10 ensures that the user whose identity is authenticated is the same person taking his/her temperature with the authenticated temperature sensing device 600.

In one such embodiment as shown in FIG. 4, the image recognition system 400 may view the temperature sensing device 600 as being located in the user's mouth (or armpit) and may visually authenticate the identity of the sensing device 600 prior to triggering the device 600 to take temperature measurements. At the same time (preferably simultaneously), the facial recognition system 404 may visually authenticate the user's identity. Once the identity of both the user Un and the temperature sensing device 600 are recognized and authenticated, the system 10 may trigger the sensing device 600 to take one or more temperature reading and communicate the readings to the application 200. The system 10 may then correlate the user's authenticated identity, the sensing device's unique identifier and the temperature readings together.

In some embodiments, the image recognition system 400 may visually recognize a unique visual identifier on the outer surface of the temperature sensing device 600 (e.g., on the underside of the sensing device's base that is viewable by the camera 302 when the device 600 is placed in the mouth of the user). In some embodiments, the unique visual identifier is a passive identifier such as, without limitation, a serial number, a QR code, a bar code, other types of identifiers and/or any combination thereof.

To provide a general understanding of this procedure, a summary of steps is described below.
1. First, the user Un launches the application 200 on the mobile device 300 and pairs the mobile device 300 with the temperature sensing device 600. This is shown in FIG. 6.
2. The mobile application 200 communicates with the sensing device 600 and acquires its identifier (e.g., serial number);
3. Next, the user Un places the temperature sensing device 600 into his/her mouth (or under his/her armpit). See FIG. 5.
4. The user Un then holds the mobile device 300 with its camera 302 pointed towards his/her face (see FIG. 2). See FIG. 5. In some embodiments, the user Un may be required to maintain this position for up to 15 seconds while this entire process runs.
5. Next, the user Un instructs the application 200 to take a temperature reading (note that this may be triggered automatically by the application 200 once it confirms that it can sufficiently view the user's face and/or the sensing device 600);
6. Next, the facial recognition system 404 views the user's face and identifies the user's identity;
7. Simultaneously, the image recognition system 400 views the visual identifier on the temperature sensing device 600 and authenticates it as the same device 600 associated with the device's identifier;
8. Upon visually identifying the user Un and the device 600, the application 200 triggers the temperature sensing device 600 to take one or more temperature readings;
8. Upon taking the temperature readings, the device 600 communicates the readings to the application 200 and the application 200 determines whether or not the user Un has an elevated body temperature (e.g., a fever).
9. The system 10 then provides this information to the user Un (e.g., on the GUI of the application 200 as shown in FIG. 7) and/or relays the screening information to any applicable entities for decision making purposes (e.g., whether or not to grant the user Un access to a particular venue). Note that in this case, the screening information may include the authenticated user's identity, the sensing device's identity, the user's body temperature, and the date and time of the temperature reading.

In some embodiments, the image recognition system 400 may visually recognize a unique active visual identifier on the outer surface of the temperature sensing device 600 (e.g., on the underside of the sensing device's base that is viewable by the camera 302 when the device 600 is placed in the mouth of the user). In some embodiments, the temperature sensing device 600 includes a unique active visual identifier such as an LED on an outer surface of the device 600 that when triggered, may release a unique and identifiable burst of light (a light identifier). The burst of light may be at a particular frequency (color), for a particular duration, may comprise a sequence of bursts each for a particular duration and/or at a particular frequency, and/or include any other unique characteristic that may be identified by the image recognition system 400. This may be used to further authenticate the temperature sensing device 600 during use.

In some embodiments, each temperature sensing device 600 is assigned both a unique light identifier and a unique digital identifier (e.g., a serial number). The system 10 may first identify the sensing device 600 using the device's digital identifier (e.g., during pairing of the mobile device 300 to the sensing device 600), and then visually identify the sensing device 600 using the light identifier just prior to triggering the measurement. In this way, the system 10 confirms that the temperature sensing device 600 taking the temperature readings (and sending them to the application 200) and the temperature device 600 visually identified as in the user's mouth are one in the same.

To provide a general understanding of this procedure, a summary of steps is described below.
1. First, the user Un launches the application 200 on the mobile device 300 and pairs the mobile device 300 with the temperature sensing device 600. This is shown in FIG. 6.
2. The mobile application 200 communicates with the sensing device 600 and acquires its identifier (e.g., serial number);
3. Next, the user Un places the temperature sensing device 600 into his/her mouth (or under his/her armpit). See FIG. 5.
4. The user Un then holds the mobile device 300 with its camera 302 pointed towards his/her face (see FIG. 2). See FIG. 5. In some embodiments, the user Un may be required to maintain this position for up to 15 seconds while this entire process runs.
5. Next, the user Un instructs the application 200 to take a temperature reading (note that this may be triggered automatically by the application 200 once it confirms that it can sufficiently view the user's face);
6. Next, the facial recognition system 400 views the user's face and identifies the user's identity;
7. Upon authenticating the identity of the user Un, the application 200 triggers the temperature sensing device 600 to release its light identifier and the application 200 visually recognizes the identifier and compares it to the sensing device's identifier;
8. Upon determining that light identifier correlates with the identifier, and therefore that the device 600 viewed by the camera 302 is the same device 600 paired with the mobile device 300, the application 200 triggers the temperature sensing device 600 to take one or more temperature readings;
9. Upon taking the temperature readings, the device 600 communicates the readings to the application 200 and the application 200 determines whether or not the user Un has an elevated body temperature (e.g., a fever).
10. The system 10 then provides this information to the user Un (e.g., on the GUI of the application 200 as shown in FIG. 7) and/or relays the screening information to any applicable entities for decision making purposes (e.g., whether or not to grant the user Un access to a particular venue).

It is understood by a person of ordinary skill in the art that the steps described above in reference to any of the above describe procedures are meant for demonstration and that not all steps may be required and that other steps not described may be performed. It is also understood that the steps may be performed in different order. It is also understood that any step(s) described in relation to any procedure may be performed in relation to any other procedure and that the resulting procedures are within the scope of the system 10.

### Interfacing with External Systems

Once the system 10 has identified and authenticated the identity of the user Un, and has successfully determined the same user's body temperature, the system 10 may provide this screening information to the user Un (via the GUI of the application 200) and/or to any other entity 700 as required (as shown in FIG. 1)

Various embodiments and details of this will be described by way of several detailed examples. The examples provided below are chosen to illustrate various embodiments and implementations of the system 10, and those of ordinary skill in the art will appreciate and understand, upon reading this description, that the examples are not limiting and that the system 10 may be used in different ways. It is also understood that details of different embodiments described in different examples may be combined in any way to form additional embodiments that are all within the scope of the system 10.

In a first example, the user Un may be an employee of a business and may have possession of an employee badge (e.g., with identifying picture and data strip) that he/she uses to gain access into the place of work. In some examples, the user Un may swipe his/her badge through a badge reader to gain access to the company facility, and/or may show the badge to a security officer who may look up the user Un on an access control system and/or visually correlate the picture on the badge with the user Un to allow access.

In this example, the badge reader system and/or the access control system may be considered external systems 700, and the system 10 may communicate with these external systems 700 as shown in FIG. 1. For example, upon successfully authenticating and acquiring screening information for the user Un, the backend system 100 may provide the badge reader system 700-1 the screening information including the user's identity, the user's body temperature data, and the date and time of the temperature reading.

In some embodiments, the external system 700 (e.g., the badge reader system 700-1) may acquire the raw screening information and make a determination regarding whether or not to allow the user Un access. For example, the badge reader system 700-1 may find the particular user Un in the system 700-1, compare the user's temperature reading to a predefined threshold temperature reading to determine whether or not the user Un has a fever, and confirm that the user's temperature reading was taken within an acceptable recent timeframe (e.g., within 2 hours prior of the user's arrival). If the user Un is confirmed to be an employee of the business, and the user's temperature reading is below the fever threshold, and the temperature reading was taken in the past 2 hours, the system 700-1 (the badge reader system) may grant the user Un access upon swiping his/her card.

If however, the user Un is not confirmed to be an employee of the business, or the user's temperature reading is greater than the fever threshold, or the time of the temperature reading is outside the acceptable time window, the badge reader system 700-1 may deny the user Un access.

In another embodiment, the system 10 may acquire (or have access to) the external system's screening parameters so that the system 10 may make the determination as to whether or not the user Un should be granted access. For example, the system 10 may have access to the external system's employee database so that the system 10 may look up and confirm the user Un as an employee, and may know the maximum body temperature threshold and/or the acceptable temperature reading time window that the external system 700 wishes to employ. In this way, the system 10 may use the external system's parameters to make the determination as to grant the user Un access or not and may convey this assessment to the external system 700. The external system 700 may receive this information and grant the user Un access accordingly. In some embodiments, the system 10 may interface with the external system 700 in real time (and/or continually) to determine if access should be granted. For example, the external system 700 may communicate with the system 10 upon the swiping of the user's badge to determine whether or not the temperature reading was taken within the specified acceptable time window with respect to the time of the badge swiping or not.

In a second example, a user Un may have a ticket to a particular event at a particular venue (e.g., a Major League Baseball game at Dodger Stadium). In some embodiments, the user Un may have an account with the entity responsible for regulating access to the venue (e.g., MLB) and the account may include information such as the identity of the user Un, etc. In some embodiments, the user's account with the system 10 may be shared with the entity such that the user Un may not be required to set up a new entity account. In this embodiment, the system 10 may interface with the entity 700 to provide it with the screening information obtained by the system 10 for the user Un prior to his/her arrival at the gate. Upon identifying themselves and/or scanning his/her ticket, the entity's access control system may look up the user Un and the user's screening information (provided in real time by the system 10) to determine whether or not the user Un is to be granted access. The integration of the system 10 with the entity may include backend integration wherein the backend platform 100 interfaces with the entity's systems, and/or frontend integration wherein the user Un may have an application on his/her mobile device 300 that allows the screening information to be provided to the entity. For instance, expanding on the above example, the user Un may have an MLB application on his/her phone that interfaces with the application 200 to receive the screening information. The MLB application may then communicate the screening information to the venue's access system for processing. In this example, the MLB application may be loaded onto the user's mobile device 300 and be enabled to interface with the application 200 (and the system 10 in general) as required.

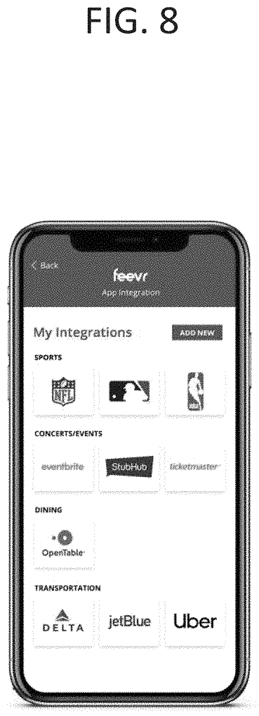

Expanding on this example, the user Un may load a variety of third-party entity applications (e.g., MLB, NFL, Live Nation, One Table, JetBlue, etc.) onto his/her mobile device 300 such that the applications may interface with the application 200 (and the overall system 10) to communicate screening information to the access control systems associated with such entities. In this way, the user Un may simply choose which application to use for a particular upcoming event, and the system 10 may interface with the associated venue as required to allow prescreening access to the user Un. This is shown in FIG. 8. As shown, in some embodiments, the third-party entity applications may be displayed on a GUI by type. In some embodiments, the third-party entity applications may communicate with the user Un directly to provide information regarding upcoming events, prescreening time window thresholds and other pertinent information.

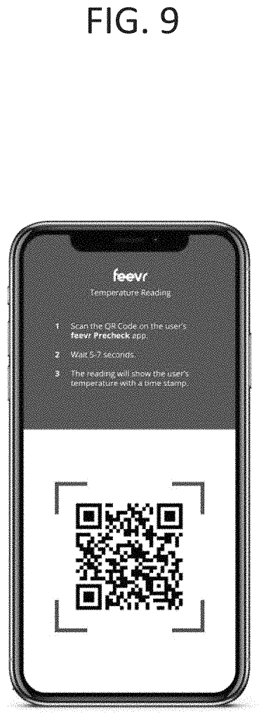

In another example, upon successfully authenticating and acquiring screening information for the user Un, the system 10 may generate a visual code (e.g., a QR code) and display the code on the screen of the user's mobile device 300. This is shown in FIG. 9. When scanned, the visual code may provide the scanner the most up to date screening information obtained for the user Un such that this information may be used to determine whether or not the user Un is to be granted access. For example, upon arriving at a restaurant that requires screening prior to access, the user Un may take his/her temperature and the system 10 may generate the corresponding QR code. The user Un may present the QR code to the restaurant who may scan the code to receive the screening information from the system 10 and grant access accordingly. In some embodiments, the restaurant may utilize an application that may be integrated with the system 10 as required. However, in some embodiments, this may not be necessary.

It is understood by a person of ordinary skill in the art that the examples provided above are meant for demonstration and that scope of the system 10 is not limited in any way by any of the examples provided. It is also understood that any details and/or aspects of any of the examples may be combined with any of the other examples to form one or more additional embodiments, all of which are within the scope of the system 10. For example, the visual QR code described in the example above in relation to gaining access to a restaurant also may be used to gain access to a place of work and/or to a sporting event.

### System Structure

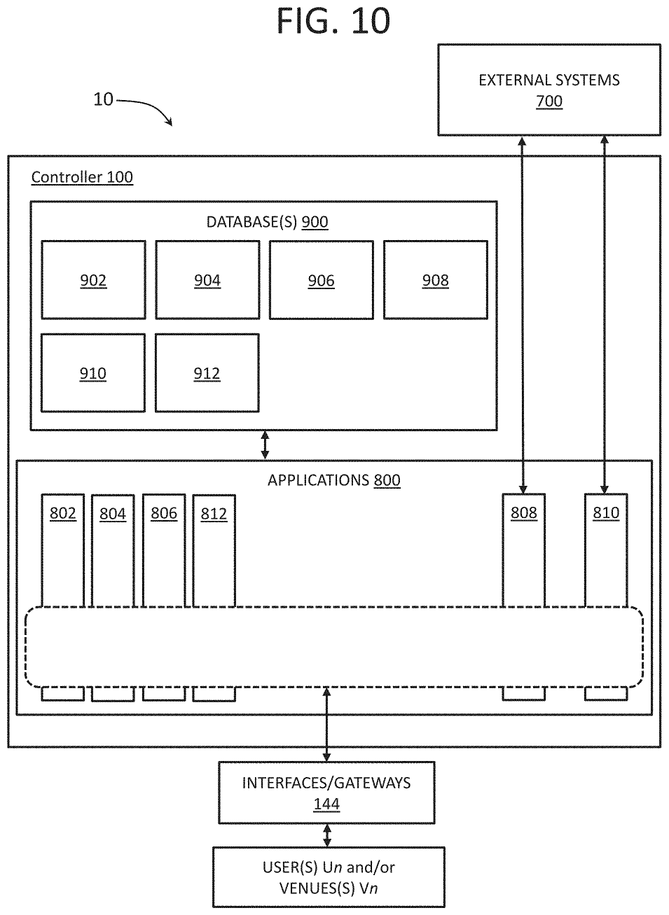

FIG. 10 shows aspects of an exemplary thermal sensing and identity authentication system 10 of FIG. 1. As shown, the system 10 and backend system 100 comprises various internal applications 800 and one or more databases 900, described in greater detail below. The internal applications 800 may generally interact with the one or more databases 900 and the data stored therein.

The database(s) 900 may comprise one or more separate or integrated databases, at least some of which may be distributed. The database(s) 900 may be implemented in any manner, and, when made up of more than one database, the various databases need not all be implemented in the same way. It should be appreciated that the system is not limited by the nature or location of database(s) 900 or by the manner in which they are implemented.

Each of the internal applications 800 may provide one or more services via an appropriate interface. Although shown as separate applications 800 for the sake of this description, it is appreciated that some or all of the various applications 800 may be combined. The various applications 800 may be implemented in any manner and need not all be implemented in the same way (e.g., using the same software languages, interfaces or protocols).

In some embodiments, the applications 800 may include one or more of the following applications 800:
1. Image recognition application(s) 802. Note that the image recognition application 802 may correlate with image recognition application 402. This application 802 may interface with any other elements of the system 10 including the image recognition database 902.
2. Facial recognition application(s) 804. Note that the facial recognition application 804 may correlate with image recognition application 404.
3. Device driver application(s) 806. These drivers enable the system 10 to interface with and control various devices such as, without limitation, the temperature sensing device 600. This application 804 may interface with any other elements of the system 10 including the image recognition database 904.
4. Data input application(s) 808. This application may receive any type of input data from any applicable system and/or element such as the application 200, the mobile device 300, the temperature sensing device 600, the external systems 700, any other system and/or element and any combination thereof.
5. Data output applications(s) 810. This application may output any type of output data to any applicable system and/or element such as the application 200, the mobile device 300, the temperature sensing device 600, the external systems 700, any other system and/or element and any combination thereof.
6. Data reporting application(s) 812. This application may generate any type of report regarding the use and/or functionalities of the system 10 including measurement data, screening information, historical data, any other types of data and/or information and any combination thereof.

The applications 800 also may include other applications and/or auxiliary applications (not shown). Those of ordinary skill in the art will appreciate and understand, upon reading this description, that the above list of applications is meant for demonstration and that the system 10 may include other applications that may be necessary for the system 10 to generally perform its functionalities as described in this specification. In addition, as should be appreciated, embodiments or implementations of the system 10 need not include all of the applications listed, and that some or all of the applications may be optional. It is also understood that the scope of the system 10 is not limited in any way by the applications that it may include.

In some embodiments, the database(s) 900 may include one or more of the following databases:
1. Image recognition database(s) 902. This database may store any data and/or other types of information related to and/or required by the image recognition application 802. For example, the database 902 may include information regarding the different types of objects (e.g., device 600) required for these objects to be recognized by the system 10.
2. Facial recognition database(s) 904. This database may store any data and/or other types of information related to and/or required by the facial recognition application 804. For example, the database 904 may include information regarding different target persons required for these persons to be recognized by the system 10.
3. Device driver(s) database(s) 906. This database may store any data, information, code, variables or other types of information related to and/or required by the device driver applications 804 to sufficiently control various devices (e.g., the device 600) as required by the system 10.
4. Measurement database(s) 908. This database may store any measurement data taken by the system 10. For example, this database may store temperature readings taken by the temperature sensing device 600 correlated with corresponding facial recognition data taken by the facial recognition application 804. This database also may store thermal imaging data taken by the thermal imaging system 500 as well as any other data acquired by the system 10.
5. Historical data database(s) 910. This database may store any and/or all historical data acquired by the system 10, including but not limited to, user data, temperature readings, times and dates of each reading, identity data of each user Un and/or target, any other information and any combination thereof.
7. Data report(s) database(s) 912. This database may store any reports of any kind generated by the system 10.

It is understood that the above list of databases is meant for demonstration and that the system 10 may include some or all of the databases, and also may include additional databases as required. It is also understood that the scope of the system 10 is not limited in any way by the databases that it may include.

Various applications 800 and databases 900 in the thermal sensing and identity authentication system 10 may be accessible via interface(s) 142. These interfaces 142 may be provided in the form of APIs or the like and made accessible to external users Un and/or vendors Vn via one or more gateways and interfaces 144 (e.g., via a web-based application 200 and/or a mobile application 200 running on a user's device 300).

It is understood that any aspect and/or element of any of the embodiments described herein or otherwise may be combined in any way to form new embodiments easily understood by a person of ordinary skill in the art. Those of ordinary skill in the art will appreciate and understand, upon reading this description, that embodiments hereof may provide different and/or other advantages, and that not all embodiments or implementations need have all advantages.

### Computing

The services, mechanisms, operations and acts shown and described above are implemented, at least in part, by software running on one or more computers or computer systems or devices. It should be appreciated that each user device is, or comprises, a computer system.

Programs that implement such methods (as well as other types of data) may be stored and transmitted using a variety of media (e.g., computer readable media) in a number of manners. Hard-wired circuitry or custom hardware may be used in place of, or in combination with, some or all of the software instructions that can implement the processes of various embodiments. Thus, various combinations of hardware and software may be used instead of software only.

One of ordinary skill in the art will readily appreciate and understand, upon reading this description, that the various processes described herein may be implemented by, e.g., appropriately programmed general purpose computers, special purpose computers and computing devices. One or more such computers or computing devices may be referred to as a computer system.

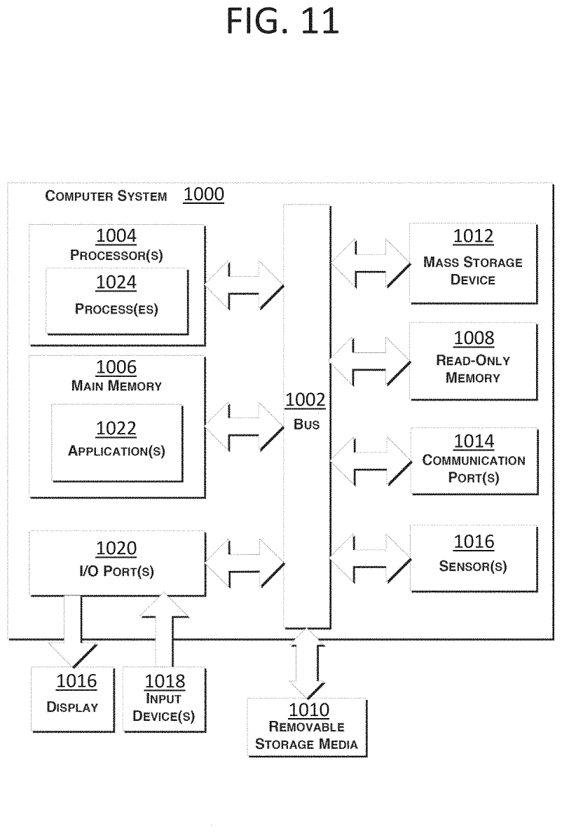

FIG. 11 is a schematic diagram of a computer system 1000 upon which embodiments of the present disclosure may be implemented and carried out.

According to the present example, the computer system 1000 includes a bus 1002 (i.e., interconnect), one or more processors 1004, one or more communications ports 1014, a main memory 1010, removable storage media 1010, read-only memory 1008, and a mass storage 1012. Communication port(s) 1014 may be connected to one or more networks by way of which the computer system 1000 may receive and/or transmit data.

As used herein, a “processor” means one or more microprocessors, central processing units (CPUs), computing devices, microcontrollers, digital signal processors, or like devices or any combination thereof, regardless of their architecture. An apparatus that performs a process can include, e.g., a processor and those devices such as input devices and output devices that are appropriate to perform the process.

Processor(s) 1004 can be (or include) any known processor, such as, but not limited to, an Intel® Itanium® or Itanium 2® processor(s), AMD® Opteron® or Athlon MP® processor(s), or Motorola® lines of processors, and the like. Communications port(s) 1014 can be any of an RS-232 port for use with a modem-based dial-up connection, a 10/100 Ethernet port, a Gigabit port using copper or fiber, or a USB port, and the like. Communications port(s) 1014 may be chosen depending on a network such as a Local Area Network (LAN), a Wide Area Network (WAN), a CDN, or any network to which the computer system 1000 connects. The computer system 1000 may be in communication with peripheral devices (e.g., display screen 1110, input device(s) 1018) via Input/Output (I/O) port 1020. Some or all of the peripheral devices may be integrated into the computer system 1000, and the input device(s) 1018 may be integrated into the display screen 1110 (e.g., in the case of a touch screen).

Main memory 1010 can be Random Access Memory (RAM), or any other dynamic storage device(s) commonly known in the art. Read-only memory 1008 can be any static storage device(s) such as Programmable Read-Only Memory (PROM) chips for storing static information such as instructions for processor(s) 1004. Mass storage 1012 can be used to store information and instructions. For example, hard disks such as the Adaptec® family of Small Computer Serial Interface (SCSI) drives, an optical disc, an array of disks such as Redundant Array of Independent Disks (RAID), such as the Adaptec® family of RAID drives, or any other mass storage devices may be used.

Bus 1002 communicatively couples processor(s) 1004 with the other memory, storage and communications blocks. Bus 1002 can be a PCI/PCI-X, SCSI, a Universal Serial Bus (USB) based system bus (or other) depending on the storage devices used, and the like. Removable storage media 1010 can be any kind of external hard-drives, floppy drives, IOMEGA® Zip Drives, Compact Disc-Read Only Memory (CD-ROM), Compact Disc-Re-Writable (CD-RW), Digital Versatile Disk-Read Only Memory (DVD-ROM), etc.

Embodiments herein may be provided as one or more computer program products, which may include a machine-readable medium having stored thereon instructions, which may be used to program a computer (or other electronic devices) to perform a process. As used herein, the term “machine-readable medium” refers to any medium, a plurality of the same, or a combination of different media, which participate in providing data (e.g., instructions, data structures) which may be read by a computer, a processor, or a like device. Such a medium may take many forms, including but not limited to, non-volatile media, volatile media, and transmission media. Non-volatile media include, for example, optical or magnetic disks and other persistent memory. Volatile media include dynamic random access memory, which typically constitutes the main memory of the computer. Transmission media include coaxial cables, copper wire and fiber optics, including the wires that comprise a system bus coupled to the processor. Transmission media may include or convey acoustic waves, light waves and electromagnetic emissions, such as those generated during radio frequency (RF) and infrared (IR) data communications.

The machine-readable medium may include, but is not limited to, floppy diskettes, optical discs, CD-ROMs, magneto-optical disks, ROMs, RAMs, erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments herein may also be downloaded as a computer program product, wherein the program may be transferred from a remote computer to a requesting computer by way of data signals embodied in a carrier wave or other propagation medium via a communication link (e.g., modem or network connection).

Various forms of computer readable media may be involved in carrying data (e.g. sequences of instructions) to a processor. For example, data may be (i) delivered from RAM to a processor; (ii) carried over a wireless transmission medium; (iii) formatted and/or transmitted according to numerous formats, standards or protocols; and/or (iv) encrypted in any of a variety of ways well known in the art.

A computer-readable medium can store (in any appropriate format) those program elements that are appropriate to perform the methods.

As shown, main memory 1010 is encoded with application(s) 1022 that support(s) the functionality as discussed herein (an application 1022 may be an application that provides some or all of the functionality of one or more of the mechanisms described herein). Application(s) 1022 (and/or other resources as described herein) can be embodied as software code such as data and/or logic instructions (e.g., code stored in the memory or on another computer readable medium such as a disk) that supports processing functionality according to different embodiments described herein.

During operation of one embodiment, processor(s) 1004 accesses main memory 1010 via the use of bus 1002 in order to launch, run, execute, interpret or otherwise perform the logic instructions of the application(s) 1022. Execution of application(s) 1022 produces processing functionality of the service(s) or mechanism(s) related to the application(s). In other words, the process(es) 1024 represents one or more portions of the application(s) 1022 performing within or upon the processor(s) 1004 in the computer system 1000.

It should be noted that, in addition to the process(es) 1024 that carries(carry) out operations as discussed herein, other embodiments herein include the application 1022 itself (i.e., the un-executed or non-performing logic instructions and/or data). The application 1022 may be stored on a computer readable medium (e.g., a repository) such as a disk or in an optical medium. According to other embodiments, the application 1022 can also be stored in a memory type system such as in firmware, read only memory (ROM), or, as in this example, as executable code within the main memory 1010 (e.g., within Random Access Memory or RAM). For example, application 1022 may also be stored in removable storage media 1010, read-only memory 1008, and/or mass storage device 1012.

Those skilled in the art will understand that the computer system 600 can include other processes and/or software and hardware components, such as an operating system that controls allocation and use of hardware resources.

As discussed herein, embodiments of the present invention include various steps or operations. A variety of these steps may be performed by hardware components or may be embodied in machine-executable instructions, which may be used to cause a general-purpose or special-purpose processor programmed with the instructions to perform the operations. Alternatively, the steps may be performed by a combination of hardware, software, and/or firmware. The term “module” refers to a self-contained functional component, which can include hardware, software, firmware or any combination thereof.

One of ordinary skill in the art will readily appreciate and understand, upon reading this description, that embodiments of an apparatus may include a computer/computing device operable to perform some (but not necessarily all) of the described process.

Embodiments of a computer-readable medium storing a program or data structure include a computer-readable medium storing a program that, when executed, can cause a processor to perform some (but not necessarily all) of the described process.

Where a process is described herein, those of ordinary skill in the art will appreciate that the process may operate without any user intervention. In another embodiment, the process includes some human intervention (e.g., a step is performed by or with the assistance of a human).

As used in this description, the term “portion” means some or all. So, for example, “A portion of X” may include some of “X” or all of “X”. In the context of a conversation, the term “portion” means some or all of the conversation.

As used herein, including in the claims, the phrase “at least some” means “one or more,” and includes the case of only one. Thus, e.g., the phrase “at least some ABCs” means “one or more ABCs”, and includes the case of only one ABC.

As used herein, including in the claims, the phrase “based on” means “based in part on” or “based, at least in part, on,” and is not exclusive. Thus, e.g., the phrase “based on factor X” means “based in part on factor X” or “based, at least in part, on factor X.” Unless specifically stated by use of the word “only”, the phrase “based on X” does not mean “based only on X.”

As used herein, including in the claims, the phrase “using” means “using at least,” and is not exclusive. Thus, e.g., the phrase “using X” means “using at least X.” Unless specifically stated by use of the word “only”, the phrase “using X” does not mean “using only X.”

In general, as used herein, including in the claims, unless the word “only” is specifically used in a phrase, it should not be read into that phrase.

As used herein, including in the claims, the phrase “distinct” means “at least partially distinct.” Unless specifically stated, distinct does not mean fully distinct. Thus, e.g., the phrase, “X is distinct from Y” means that “X is at least partially distinct from Y,” and does not mean that “X is fully distinct from Y.” Thus, as used herein, including in the claims, the phrase “X is distinct from Y” means that X differs from Y in at least some way.

As used herein, including in the claims, a list may include only one item, and, unless otherwise stated, a list of multiple items need not be ordered in any particular manner. A list may include duplicate items. For example, as used herein, the phrase “a list of XYZs” may include one or more “XYZs”.

It should be appreciated that the words “first” and “second” in the description and claims are used to distinguish or identify, and not to show a serial or numerical limitation. Similarly, the use of letter or numerical labels (such as “(a)”, “(b)”, and the like) are used to help distinguish and/or identify, and not to show any serial or numerical limitation or ordering.

No ordering is implied by any of the labeled boxes in any of the flow diagrams unless specifically shown and stated. When disconnected boxes are shown in a diagram the activities associated with those boxes may be performed in any order, including fully or partially in parallel.

While the invention has been described in connection with what is presently considered to be the most practical and preferred embodiments, it is to be understood that the invention is not to be limited to the disclosed embodiments, but on the contrary, is intended to cover various modifications and equivalent arrangements included within the spirit and scope of the appended claims.

1. (canceled)

2. A method of determining the identity and body temperature of a user, the method comprising:
(A) storing first identity authentication information relating to a first user;
(B) providing a mobile device including a facial recognition system;
(B) providing a temperature sensing device;
(C) using the facial recognition system to acquire second identity authentication information relating to the first user;
(D) comparing the first identity authentication information and the second identity authentication information to confirm an identity of the first user;
(E) upon a confirmation of the identity of the first user, then:
(F) using the temperature sensing device to determine a first body temperature of the first user;
(G) using the mobile device to acquire from the temperature sensing device information based on the first body temperature of the first user;
(H) using the mobile device, analyzing the information based on the first body temperature of the first user to information to determine an elevated or a non-elevated first body temperature of the first user.

3. The method of claim 2 further comprising:
(I) using the mobile device, communicating the information based on the first body temperature of the first user to a cloud platform.

4. The method of claim 2 further comprising:
(I) displaying the elevated or the non-elevated first body temperature of the first user on a display of the mobile device.

5. The method of claim 2 further comprising:
(I) using the mobile device to record a date and time of the determination of the first body temperature of the first user.

6. The method of claim 2 wherein using the temperature sensing device to determine a first body temperature of the first user in (F) includes using the mobile device to trigger the temperature sensing device to take a temperature reading.

7. The method of claim 2 further comprising:
(E)(1) using the mobile device to acquire an identifier of the temperature sensing device.

8. The method of claim 7 further comprising:
(E)(2) using the mobile device, authenticating the identifier of the temperature sensing device.

9. The method of claim 8 further comprising:
(E)(3) using the facial recognition system to confirm that the temperature sensing device is located in the first user's mouth.

10. The method of claim 2 further comprising:
(E)(1) instructing the first user to place the temperature sensing device into his/her mouth.

11. The method of claim 2 wherein the mobile device is adapted to wirelessly communicate with the temperature sensing device.

12. A system for determining the body temperature of a user, the system comprising:
an image recognition system;
a temperature sensing device;
a cloud platform; and
an electronic communication device in communication with the image recognition system, the temperature sensing device, and the cloud platform;
wherein the image recognition system is adapted to authenticate an identity of a user, the temperature sensing device is adapted to determine a first body temperature of the user, and the electronic communication device is adapted to communicate the identity and the first body temperature of the user to the cloud platform.
